(19) United States
(12) Patent Application Publication
(10) Pub. No.: US 2011/0105693 A1
(43) Pub. Date: May 5, 2011

(54) CATIONIC TRANSITION-METAL ARENE CATALYSTS

(75) Inventors: Kamaluddin Abdur-Rashid, Mississauga (CA); Dino Amoroso, Binbrook (CA); Christine Sui-Seng, Toronto (CA); Wenli Jia, Toronto (CA); Charles Ewart, Toronto (CA)

(73) Assignee: KANATA CHEMICAL TECHNOLOGIES INC., Toronto, ON (CA)

(21) Appl. No.: 12/990,335

(22) PCT Filed: May 1, 2009
(86) PCT No.: PCT/CA2009/000568

§ 371 Date: Dec. 1, 2010

Related U.S. Application Data
(60) Provisional application No. 61/049,580, filed on May 1, 2008.

(51) Int. Cl. C08F 112/14; C07F 15/00; C07C 37/055; C07D 209/08

(52) U.S. Cl. 525/333.5; 546/4; 556/7; 556/13; 568/814; 548/490

(57) ABSTRACT

Disclosed are cationic ruthenium arene complexes of Formula (I): [Ru(D-Z¹—NHR¹)(Ar)(LB)ₙ]^{r+}[Y⁻]ᵣ, wherein Ar is optionally substituted aryl, D-Z¹—NHR¹ is a coordinated bidentate ligand wherein D, Z¹, R¹ and R² are as defined herein, and where R¹ and Ar, or R² and Ar may be linked together, n is 0 or 1, r is 1 or 2, LB is a neutral Lewis base, and Y is a non-coordinating anion. The complexes are active catalysts for reduction reactions, including the transfer-hydrogenation of carbon-oxygen (C═O) and carbon-nitrogen (C═N) double bonds.

### FIELD OF THE DISCLOSURE

The present disclosure relates to the field of catalytic organic synthesis transformations in which a catalytic system comprising a cationic transition-metal arene complex is used, for example for the transfer hydrogenation or reduction of compounds containing a carbon-heteroatom (C═O, C═N) double bond.

### BACKGROUND OF THE DISCLOSURE

Ruthenium arene complexes (1) incorporating chelating tosylated diamine ligands are a well-understood class of highly active and selective transfer hydrogenation catalysts. These complexes were originally reported as competent catalysts in early work by Noyori et al. wherein it was disclosed that such complexes could reduce ketones/aldehydes¹ and imines², for the preparation of alcohols and amines respectively, including chiral compounds, under transfer hydrogenation conditions. Indeed, this family of catalysts and materials derived through their application in transfer hydrogenation have been extensively protected.³

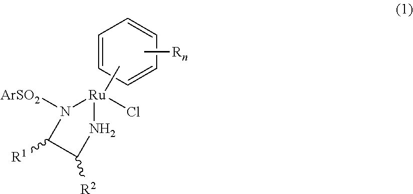

Ruthenium arene complexes have also been described as useful catalysts for enantioselective Michael addition⁴ or 1,4-addition.⁵ Moreover, supported (i.e. on polymer) arene complexes of ruthenium have been claimed as valuable catalysts for a range of catalytic transformations including olefin metathesis, hydrogenation and alkyne cyclization.⁶ Tethered arene complexes (i.e. where the arene and diamines are linked via a tether) have also been reported which are similarly useful in a range of processes.⁷

### SUMMARY OF THE DISCLOSURE

The transfer hydrogenation of ketones, aldehydes and imines has been successfully and advantageously performed using cationic salts of certain neutral Ru(II) complexes. The cationic complexes were prepared by treatment of the neutral precursors with anion abstracting agents. The resulting complexes were air and moisture stable. Solutions could be prepared and handled in air with no obvious signs of decay. The activity of the cationic complexes matched that of the neutral precursors. In several cases, the cationic derivatives gave products with improved enantiomeric excess relative to the neutral congener.

Accordingly, the present disclosure includes a compound of Formula I:

[Ru(D-Z¹—NHR¹)(Ar)(LB)ₙr[Y⁻]ᵣ (I)

wherein
Ar is optionally substituted aryl, wherein the optional substituents are selected from one or more of, suitably one to four, more suitably one to two, halo, C₁₋₆alkyl, fluoro-substituted-C₁₋₆alkyl, C₂₋₆alkenyl, C₂₋₆alkynyl, aryl and fluoro-substituted aryl, and Ar is optionally linked to a polymeric support;
LB is any neutral Lewis base;
Y is any non-coordinating anion;
n is 0 or 1;
r is 1 or 2;
D-Z¹—NHR¹ is a coordinated bidentate ligand in which
Z¹ is C₂-C₇alkylene, C₄-C₁₀cycloalkylene, metallocenediyl, C₆-C₂₂arylene or combinations of one or more of, suitably one to four, more suitably one to two, C₂-C₇alkylene, C₄-C₁₀cycloalkylene, metallocenediyl and C₆-C₂₂arylene, said C₂-C₇alkylene, C₄-C₁₀cycloalkylene, metallocenediyl and C₆-C₂₂arylene groups being optionally substituted, wherein the optional substituents are selected from one or more of, suitably one to four, more suitably one to two, halo, C₁₋₆alkyl, fluoro-substituted-C₁₋₆alkyl, C₂₋₆alkenyl, C₂₋₆alkynyl, aryl and fluoro-substituted aryl;
D is NR², OR², SR², SeR² or TeR²;
R² is H, S(O)₂R³, P(O)(R³)₂, C(O)R³, C(O)N(R³)₂ or C(S)N(R³)₂; and
R¹ and R³, are simultaneously or independently H, C₂₋₈alkenyl, C₃₋₁₀cycloalkyl or aryl, said latter 4 groups being optionally substituted wherein the optional substituents are selected from one or more of, suitably one to four, more suitably one to two, halo, C₁₋₆alkyl, fluoro-substituted-C₁₋₆alkyl, C₂₋₆alkenyl, C₂₋₆alkynyl, aryl and fluoro-substituted aryl,
or R¹ and Ar, or R² and Ar, are linked via Z²,
wherein Z² is as defined as Z¹ above, and wherein one or more carbon atoms,suitably one to four, more suitably one to two, in Z² is optionally replaced with —O—, —S—, —C(═O)—, —S(═O)—, —S(═O)₂—, —PR³—, —P(═O)R³—, NH or NR³.

The present disclosure is also directed to processes for organic synthesis reactions using the compounds of Formula I. For example the compounds of Formula I are useful as catalysts for transfer hydrogenations, hydrogenations, Michael additions, 1,4-additions, olefin metathesis and alkyne cyclizations. The present disclosure therefore includes methods of performing these reactions comprising contacting a compound of the Formula I with the appropriate starting reagent(s) and reacting under conditions sufficient to perform the reaction.

In a particular embodiment of the present disclosure there is included a process for the reduction of compounds comprising one or more carbon-oxygen (C═O) or carbon-nitrogen (C═N) double bonds, to the corresponding hydrogenated alcohol or amine, comprising contacting a compound comprising the C═O or C═N double bond(s) with a compound of the Formula I under transfer hydrogenation conditions.

In an embodiment of the invention, the compound comprising one or more carbon-oxygen (C═O) or carbon-nitrogen (C═N) double bonds is a compound of Formula (III):

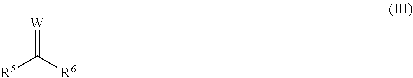

wherein,
W is selected from NR⁷, (NR⁷R⁸)⁺Q⁻ and O;
R⁵ and R⁶ are simultaneously or independently selected from H, aryl, C₁₋₂₀alkyl, C₂₋₂₀alkenyl, C₃₋₂₀cycloalkyl and heteroaryl, said latter 5 groups being optionally substituted;
R⁷ and R⁸ are independently or simultaneously selected from H, OH, C₁₋₂₀alkoxy, aryloxy, C₁₋₂₀alkyl, C₂₋₂₀alkenyl, C₃₋₂₀cycloalkyl and aryl, said latter 6 groups being optionally substituted;
or
one or more of R⁵ to R⁸ are linked to form, together with the atoms to which they are attached, an optionally substituted ring system; and
Q⁻ represents a counter anion,
wherein heteroaryl is a mono- or bicyclic heteroaromatic group containing from 5 to 10 atoms, of which 1-3 atoms is optionally a heteroatom selected from S, O and N, and wherein the optional substituents are selected from one or more of, suitably one to four, more suitably one to two, halo, OH, NH₂, OR⁹, NR⁹₂ and R⁹, in which R⁹ is selected from C₁₋₆alkyl, C₂₋₆alkenyl and aryl and one or more of, suitably one to four, more suitably one to two, the carbon atoms in the alkyl, alkenyl and cycloalkyl groups is optionally replaced with a heteroatom selected from the group consisting of O, S, N, P and Si.

Reduction of compounds of Formula III using a compound of the Formula I according to the process described above provides the corresponding hydrogenated compounds of Formula (IV):

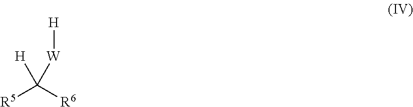

wherein W, R⁵ and R⁶ are defined as in Formula (III).

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will now be described in greater detail with reference to the attached drawings in which:

FIG. 1 is an X-ray crystal structure of [(p-cymene)Ru(TsDPEN)(pyridine)]BF₄. Some hydrogen atoms, a CH₂Cl₂ molecule and a BF₄ counteranion have been omitted for clarity;

FIG. 2 is an X-ray crystal structure of [(p-cymene)Ru(TsDPEN)]BF₄. Most hydrogen atoms and a BF₄ counteranion have been omitted for clarity;

FIG. 3 is a graph illustrating the effect of time and base on conversion and enantiomeric excess in transfer hydrogenation of acetophenone in i-PrOH where (R,R)—BF₄=[(p-cymene)Ru(R,R-TsDPEN)]BF₄; (S,S)(pyr)-BF₄=[(p-cymene)Ru(S,S-TsDPEN)(pyridine)]BF₄;

FIG. 4 is a graph showing the effect of triethylamine/formic acid (TEAF) volume and co-solvent on conversion and enantiomeric excess in the transfer hydrogenation of acetophenone in TEAF after 4 h and 20 h respectively using [(p-cymene)Ru(R,R-TsDPEN)BF₄; and

FIG. 5 is a graph showing the effect of triethylamine/formic acid (TEAF) volume, co-solvent and water on conversion and e.e. in the transfer hydrogenation of acetophenone in TEAF after 4 h and 20 h respectively using [(p-cymene)Ru(R,R-TsDPEN)]BF₄ (“Top layer” and “bottom layer” indicate which layer of the biphasic mixture is analyzed since water is not miscible in the organic solvent).

### DETAILED DESCRIPTION OF THE DISCLOSURE

### (I) Definitions

The term “C₁₋ₙalkyl” as used herein means straight and/or branched chain, saturated alkyl radicals containing from one to “n” carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable n is an integer representing the largest number of carbon atoms in the alkyl radical.

The term “C₂₋ₙalkenyl” as used herein means straight and/or branched chain, unsaturated alkyl radicals containing from one to n carbon atoms and one to three double bonds, and includes (depending on the identity of n) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like, where the variable n is an integer representing the largest number of carbon atoms in the alkenyl radical.

The term “C₂₋ₙalkynyl” as used herein means straight and/or branched chain, unsaturated alkyl radicals containing from one to n carbon atoms and one to three triple bonds, and includes (depending on the identity of n) acetylenyl, 1-propynyl, 2-propynyl, 3-methylprop-1-ynyl, but-1-ynyl, but-2-ynyl, but-3-ynyl, 4-methylbut-1-ynyl, 4-methylbut-2-ynyl, 3-methylbut-1-ynyl, 2-methylpent-3-ynyl, 4-methylpent-1-ynyl, 4-methyl pent-2-ynyl, 5-methylpenta-1,3-diynyl, hexyn-1-yl and the like, where the variable n is an integer representing the largest number of carbon atoms in the alkynyl radical.

The term “C₃₋₂₀cycloalkyl” as used herein means a monocyclic, bicyclic or tricyclic saturated carbocylic group containing from three to twenty carbon atoms and includes cyclopropyl, cyclobutyl, cyclopentyl, cyclodecyl and the like.

The term “aryl” as used herein means a monocyclic, bicyclic or tricyclic aromatic ring system containing from 6 to 14 carbon atoms and at least one aromatic ring and includes phenyl, naphthyl, anthracenyl, 1,2-dihydronaphthyl, 1,2,3,4-tetrahydronaphthyl, fluorenyl, indanyl, indenyl and the like.

The term “heteroaryl” as used herein refers to a mono- or bicyclic heteroaromatic group containing at least one aromatic ring and from 5 to 10 atoms, of which 1-3 atoms is a heteromoiety selected from the group consisting of S, O and N, NH and NC₁₋₄alkyl.

The term “metallocenediyl” as used herein means a divalent metallocene containing a transition-metal and two cyclopentadienyl ligands coordinated in a sandwich structure, i. e., the two cyclopentadienyl anions are co-planar with equal bond lengths and strengths.

The suffix “ene” added on to the name of a group means that the group is divalent.

The term “divalent” as used herein means that the referenced group has at least two covalent bonds with other groups.

The term “halo” as used herein means halogen and includes chloro, flouro, bromo and iodo.

The term “fluoro-substituted” as used herein means that one or more, including all, suitably one to four, more suitably one to two, of the hydrogens on the referenced group is replaced with fluorine.

The term “optionally substituted” means unsubstituted or substituted.

The term “ring system” as used herein refers to a carbon-containing ring system, that includes monocycles, fused bicyclic and polycyclic rings, bridged rings and metalocenes. Where specified, the carbons in the rings may be substituted or replaced with heteromoieties selected from O, S, N, N—H and NC₁₋₄alkyl.

The term “stereogenic” as used herein refers to a molecule or a portion of a molecule that has a chiral center and therefore has different stereoisomers. It will also be understood by those skilled in the art that a molecule or a portion of a molecule can possess a stereogenic plane, so that the molecule possesses planar chirality.

The term “bidentate” as used herein refers to a ligand that bonds to the metal, M, via two donor sites.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Compounds of the Disclosure

Rendering the neutral arene transition-metal complexes of the present disclosure into an ionic pair dramatically alters the behaviour and properties of the original metal complex. While not wishing to be limited by theory, these changes may be borne out of changes in structure of the resulting complex, the charged nature of the newly formed ionic complex or they may be a result of qualities imparted by the new counter ion. Regardless of the origin of effect, there were great advantages gained from this approach in the present disclosure.

Removal of any ligand from the transition-metal complexes of the present disclosure had the effect of introducing a vacant coordination site (i.e. coordinatively unsaturated). In transition-metal catalysis this is often imperative for substrate binding and may indeed be rate limiting with respect to the catalytic cycle. Abstraction of an anionic ligand and substituting it with a non- or weakly-coordinating anion represents one such method for installing a vacant coordination site. In this manner, generating cationic complexes by abstraction of coordinating anionic ligands and substitution with non-coordinating anionic ligands lead to more active catalysts.

The exchange of a coordinating anionic ligand with a non-coordinating or weakly-coordinating ligand resulted in a more electrophillic, cationic metal centre. This increased electrophillicity lead to stronger binding between the metal and nucleophilic substrates. With respect to catalytic processes involving metal-substrate interactions, this has the obvious consequences and is especially beneficial in the case of weaker nucleophiles such as those with electron-withdrawing groups.

Transforming the covalent transition-metal complexes of the present disclosure into ionic salts lead to derivatives which were more stable than their parents. While not wishing to be limited by theory, increased stability is the result of the removal of electron density from the metal leading to a metal centre which is less readily oxidized. Thus, the ionic salts prepared from neutral precursors were generally more stable to oxidation under atmospheric conditions displaying greater tolerance toward oxygen and moisture and greater storage stability (i.e. shelf-life).

The solubility properties of ionic complexes were also different from their neutral precursors. Generally, ionic complexes tended to be more soluble in polar solvents and less soluble in apolar solvents. Some ionic complexes were also more soluble in aqueous solutions. That being said, the solubility of the ionic complex can be further tuned with the selection of the anion. For instance, highly fluorinated anions tended to impart a high degree of solubility in a broad range of solvents. In fact, many ionic complexes incorporating highly fluorinated anions were more soluble in nonpolar solvents than the corresponding neutral precursor while their solubility in polar solvents remained high owing to the ionic nature of the complex.

The ability to tailor solubility also afforded the ability to control the solid properties of the ionic complex. That is, polar salts could be readily precipitated with nonpolar solvents leading to higher isolated yields and more regular and controllable particle sizes. A corollary to this property is that these ionic catalysts also hold the promise of more facile removal from product mixtures—an obvious benefit when one considers the use of ionic catalysts in applications where low residual metals are imperative. In addition, by improving the solubility, the activity and selectivity improves as the cationic compounds rapidly dissolve in reaction solvents without extended periods of heating (which generally leads to partial decomposition thereby diminishing both yield and selectivity of the less soluble neutral parent compounds).

While rendering a neutral catalyst cationic holds the promise of many critical advantages, the utility of this approach is limited by competence in catalysis of the resulting ionic complex. If the derived ionic catalyst is no longer active in catalysis then the advantages described above are obviously moot. As a representative example, in the present disclosure, the cationic ruthenium catalysts were shown to be excellent transfer hydrogenation catalysts. The activity of the cationic complexes matched that of the neutral precursors and, in several cases, the cationic derivatives gave products with improved enantiomeric excess relative to the neutral congener. While not wishing to be limited by theory, this is likely due to the fact that the cationic complexes disclosed herein are more reliably and reproducibly activated prior to entering the catalytic cycle. That is to say, that while all of the complexes are subject to activation, the cationic complexes fare better in this process than the neutral analogues. The activation process, which is carried out in alcohol solvents and is often irreproducible and unpredictable, is better suited to the cationic complexes since they are soluble in the solvent system while the neutral complexes are either insoluble or moderately soluble. The poor solubility of the neutral compounds means that the activation is often incomplete and can lead to side reactions giving catalytically inactive species or active species which do not retain the desired stereoselectivity.

Accordingly, the present disclosure includes a compound of Formula I:

[Ru(D-Z¹—NHR¹)(Ar)(LB)ₙ]^{r+}[Y⁻]ᵣ (I)

wherein
Ar is optionally substituted aryl, wherein the optional substituents are selected from one or more of, suitably one to four, more suitably one to two, halo, C₁₋₆alkyl, fluoro-substituted-C₁₋₆alkyl, C₂₋₆alkenyl, C₂₋₆alkynyl, aryl and fluoro-substituted aryl, and Ar is optionally linked to a polymeric support;
LB is any neutral Lewis base;
Y is any non-coordinating anion;
n is 0 or 1;
r is 1 or 2;
D-Z¹—NHR¹ is a coordinated bidentate ligand in which
Z¹ is C₂-C₇alkylene, C₄-C₁₀cycloalkylene, metallocenediyl, C₆-C₂₂arylene or combinations of one or more of, suitably one to four, more suitably one to two, C₂-C₇alkylene, C₄-C₁₀cycloalkylene, metallocenediyl and C₆-C₂₂arylene, said C₂-C₇alkylene, C₄-C₁₀cycloalkylene, metallocenediyl and C₆-C₂₂arylene groups being optionally substituted, wherein the optional substituents are selected from one or more of, suitably one to four, more suitably one to two, halo, C₁₋₆alkyl, fluoro-substituted-C₁₋₆alkyl, C₂₋₆alkenyl, C₂₋₆alkynyl, aryl and fluoro-substituted aryl;
D is NR², OR², SR², SeR² or TeR²;
R² is H, C₁₋₂₀alkyl, S(O)₂R³, P(O)(R³)₂, C(O)R³, C(O)N(R³)₂ or C(S)N(R³)₂; and
R¹ and R³, are simultaneously or independently H, C₁₋₈alkyl, C₂₋₈alkenyl, C₃₋₁₀cycloalkyl or aryl, said latter 4 groups being optionally substituted wherein the optional substituents are selected from one or more of, suitably one to four, more suitably one to two, halo, C₁₋₆alkyl, fluoro-substituted-C₁₋₆alkyl, C₂₋₆alkenyl, C₂₋₆alkynyl, aryl and fluoro-substituted aryl,
or R¹ and Ar, or R² and Ar, are linked via Z²,
wherein Z² is as defined as Z¹ above, and wherein one or more carbon atoms, suitably one to four, more suitably one to two, in Z² is optionally replaced with —O—, —S—, —C(═O)—, —S(═O)—, —S(═O)₂—, —PR³—, —P(═O)R³—, NH or NR³.

In another embodiment of the present disclosure, Ar is optionally substituted phenyl, the optional substituents selected from one or more of, suitably one to four, more suitably one to two, halo, C₁₋₆alkyl, fluoro-substituted-C₁₋₆alkyl, C₂₋₆alkenyl, C₂₋₆alkynyl and aryl. In a further embodiment, Ar is

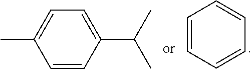

In another embodiment of the present disclosure, Ar is linked to a polymeric support. In a further embodiment, the polymer support is polystyrene. When the compound of Formula I is linked to a polymer support through Ar, the compound of Formula I is easily separated from the reaction products in organic synthesis reactions. Methods of attaching molecules to polymer supports are well-known in the art.

It is an embodiment of the present disclosure that D-Z¹—NHR¹ is a chiral coordinated bidentate amine ligand. In a further embodiment, Z¹ is C₂-C₄alkylene, C₅₋₈cycloalkylene, ferrocendiyl, phenylene, naphthylene or bisphenylene, said 6 groups being optionally substituted, wherein the optional substituents are selected from one or more of, suitably one to four, more suitably one to two, halo, C₁₋₄alkyl, fluoro-substituted-C₁₋₄alkyl, phenyl and fluoro-substituted phenyl. In another embodiment, Z¹ is optionally substituted C₂₋₄alkylene wherein the optional substituents are selected from one or two of halo, C₁₋₄alkyl, fluoro-substituted-C₁₋₄alkyl, phenyl and fluoro-substituted phenyl.

It is another embodiment of the present disclosure that D is NR². Further, it is an embodiment that R² is S(O)₂R³, P(O)(R³)₂, C(O)R³, C(O)N(R³)₂ or C(S)N(R³)₂. In another embodiment, R² is S(O)₂R³ or C(O)R³.

In another embodiment of the disclosure, D is NR², wherein R² is S(O)₂R³ or C(O)R³. Accordingly, the coordinated bidentate amine ligand is an amidoamino ligand that comprises an amido or sulfamido group donor NR² and an amino group donor NHR¹, the substituent R² representing S(O)₂R³ or C(O)R³. In a further embodiment, the groups R¹ and R³, are simultaneously or independently, H, C₁₋₆alkyl, C₂₋₆alkenyl, C₅₋₈cycloalkyl or aryl, said latter 4 groups being optionally substituted wherein the optional substituents are selected from one or more of, suitably one to four, more suitably one to two, halo, C₁₋₄alkyl, fluoro-substituted-C₁₋₄alkyl, aryl and fluoro-substituted aryl. In suitable embodiments of the present disclosure, the bidentate amine ligand is chiral and includes (1) compounds in which the amine-bearing center (NHR¹) is stereogenic, (2) compounds in which both the donor-bearing (D) and amine-bearing centers (NHR¹) are stereogenic (for example the ligand CH₃C₆H₄SO₃NCHPhCHPhNH₂). In another embodiment, R¹ and R³ are simultaneously or independently, H, C₂₋₆alkyl, C₂₋₆alkenyl, C₅₋₈cycloalkyl or phenyl, said latter four groups being optionally substituted wherein the optional substituents are selected from one or more of, suitably one to four, more suitably one to two, halo, C₁₋₄alkyl, fluoro-substituted-C₁₋₄alkyl, phenyl and fluoro-substituted phenyl. In another embodiment, R¹ is H. In another embodiment, R³ is

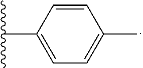

In another embodiment of the disclosure, the compounds of Formula I are compounds in which Ar is linked through Z² to R¹ and/or R³, wherein Z² is as defined as Z¹, and wherein one or more carbon atoms, suitably one to four, more suitably one to two, in Z² is optionally replaced with —O—, —S—, —C(═O)—, —S(═O)—, —S(═O)₂—, —PR³—, —P(═O)R³—, NH or NR³. In another embodiment, Z² is C₂-C₄alkylene, C₅₋₈cycloalkylene, ferrocendiyl, phenylene, naphthylene or bisphenylene, said 6 groups being optionally substituted, wherein the optional substituents are selected from one or more of, suitably one to four, more suitably one to two, halo, C₁₋₄alkyl, fluoro-substituted-C₁₋₄alkyl, phenyl and fluoro-substituted phenyl and wherein one or more carbon atoms in Z² is optionally replaced with —O—, —S—, —C(═O)—, —S(═O)—, —S(═O)₂—, —PR³—, —P(═O)R³—, NH or NR³. In another embodiment, Z² is optionally substituted C₂₋₄alkylene or optionally substituted phenylene, wherein the optional substituents are selected from one or two of halo, C₁₋₄alkyl, fluoro-substituted-C₁₋₄alkyl, phenyl and fluoro-substituted phenyl. In another embodiment, Z² is optionally substituted C₂₋₄alkylene wherein the optional substituents are selected from one or two of halo, C₁₋₄alkyl, fluoro-substituted-C₁₋₄alkyl, phenyl and fluoro-substituted phenyl. In another embodiment, Z² is optionally substituted propylene wherein the optional substituents are selected from one or two of halo, C₁₋₄alkyl, fluoro-substituted-C₁₋₄alkyl, phenyl and fluoro-substituted phenyl. In another embodiment, Z² is propylene.

In an embodiment of the disclosure, LB is any suitable neutral Lewis base, for example any neutral two electron donor, for example acetonitrile, DMF or pyridine.

In another embodiment of the disclosure, Y is any weakly or non-coordinating counter anion, including, but not limited to, OTf, BF₄, PF₆, B(C₁₋₆alkyl)₄, B(fluoro-substituted-C₁₋₆alkyl)₄ or B(aryl)₄ wherein aryl is unsubstituted or substituted one or more times, optionally one to five times, optionally one to three times, with fluoro, C₁₋₄alkyl or fluoro-substituted C₁₋₄alkyl. In an embodiment of the present disclosure, Y is a weakly coordinating or non-coordinating anion. In another embodiment, Y is OTf, BF₄⁻, CF₃SO₃⁻, PF₆⁻, B(C₆F₅)₄⁻, B[3,5-(CF₃)₂C₆H]₄⁻ or

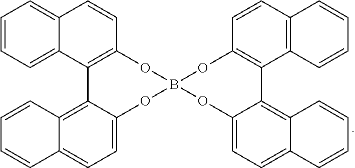

In another embodiment of the present disclosure, the compound of Formula I is

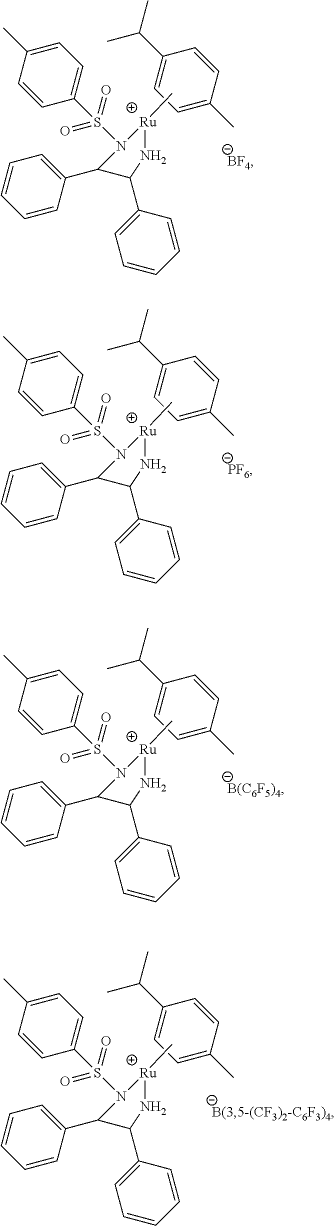

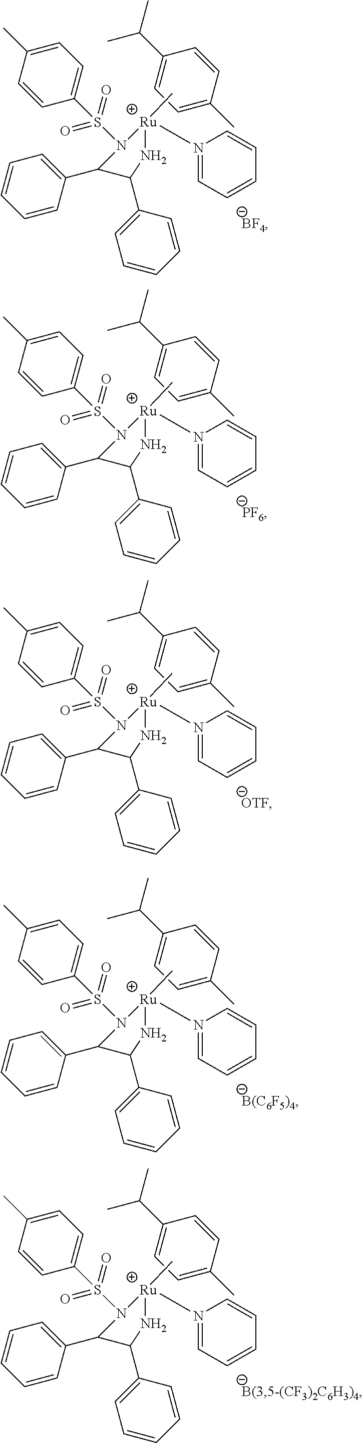

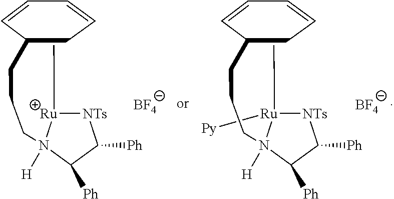

In a general way, the neutral precursors corresponding to the compounds of Formula (I) can be prepared and isolated prior to their use in the process according to the general methods described in the literature or using the methods described herein. In an embodiment of the disclosure, formation of the cationic catalyst is performed by reacting the neutral complex with an anion-abstracting agent, suitably in an inert atmosphere at ambient or room temperature. In general, the halide, suitably the chloride, bound to the neutral complex is abstracted by treatment with a salt of a non-coordinated anion (i.e. one which does not formally bond to or share electrons with the metal center in a typical covalent bond). This leads to formation of a salt complex comprised of a formally cationic transition-metal complex and the associated, non- or weakly-coordinating anion. In another embodiment, the formation of the compound of the Formula I is via a procedure where the precursor to the neutral complexes, for example [RuCl₂(p-cymene)]₂, is first rendered cationic by treatment with a salt of a non-coordinated anion and then treated with the appropriate diamine ligand to generate the compounds of Formula I. Also, a one-pot procedure can also be envisioned where all of the components are combined to generate the cationic transition-metal diamine complexes. Coordinatively saturated complexes can be prepared by treating the coordinatively unsaturated materials with coordinating Lewis bases (for e.g. pyridine).

### (III) Processes of the Disclosure

The present disclosure further includes a process for preparing a compound of Formula I comprising combining a precursor ruthenium compound, an anion abstracting agent, a compound of the Formula D-Z¹—NHR¹ wherein D, Z¹ and R¹ are as defined above, and optionally a base and reacting under conditions to form the compound of Formula I and optionally isolating the compound of Formula I. In an embodiment of the disclosure, the precursor ruthenium compound has the Formula [Ru(ligand)]₂, wherein ligand is any displaceable ligand, for example, p-cymene. In yet another embodiment, the anion abstracting agent is a salt of a non-coordinating anion. In a further embodiment, the base is an organic base, such as an amine, for example triethylamine. In another embodiment, the conditions to form the compound of Formula I comprise reacting at a temperature of about 50° C. to about 100° C. in a suitable solvent, for example THF, for about 30 minutes to 48 hours, following by cooling to room temperature. In an embodiment of the disclosure, the compound of Formula I is isolated by filtration and evaporation of the filtrate to provide the compound of Formula I.

The present disclosure also relates to a process for performing organic synthesis reactions using the compounds of Formula I. For example the compounds of Formula I are useful as catalysts for transfer hydrogenations, hydrogenations, Michael additions, 1,4-additions, olefin metathesis and alkyne cyclizations. The present disclosure therefore includes methods of performing these reactions comprising contacting a compound of the Formula I with the appropriate starting reagent(s) and reacting under conditions sufficient to perform the reaction. Such conditions would be known to a person skilled in the art.

In a particular embodiment of the present disclosure there is included a process for the reduction of compounds comprising one or more carbon-oxygen (C═O) or carbon-nitrogen (C═N) double bonds, to the corresponding hydrogenated alcohol or amine, comprising contacting a compound comprising the C═O or C═N double bond(s) with a compound of the Formula I under transfer hydrogenation conditions.

In an embodiment of the invention, the compound comprising one or more carbon-oxygen (C═O) or carbon-nitrogen (C═N) double bond(s) is a compound of formula (III):

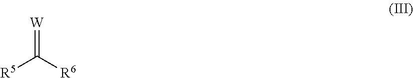

wherein,
W is selected from NR⁷, (NR⁷R⁸)⁺Q⁻ and O;
R⁵ and R⁶ are simultaneously or independently selected from H, aryl, C₁₋₂₀alkyl, C₂₋₂₀alkenyl, C₃₋₂₀cycloalkyl and heteroaryl, said latter 5 groups being optionally substituted;
R⁷ and R⁸ are independently or simultaneously selected from H, OH, C₁₋₂₀alkoxy, aryloxy, C₁₋₂₀alkyl, C₂₋₂₀alkenyl, C₃₋₂₀cycloalkyl and aryl, said latter 6 groups being optionally substituted;
or
one or more of R⁵ to R⁸ are linked to form, together with the atoms to which they are attached, an optionally substituted ring system; and Q⁻ represents a counteranion,
wherein heteroaryl is a mono- or bicyclic heteroaromatic group containing from 5 to 10 atoms, of which 1-3 atoms is optionally a heteroatom selected from S, O and N, and wherein the optional substituents are selected from halo, OH, NH₂, OR⁹, NR⁹₂ and R⁹, in which R⁹ is selected from C₁₋₆alkyl, C₂₋₆alkenyl and aryl and one or more, suitably one to four, more suitably one to two, of the carbon atoms in the alkyl, alkenyl and cycloalkyl groups is optionally replaced with a heteromoiety selected from O, S, N, NH and NC₁₋₄alkyl.

Reduction of compounds of Formula III using a compound of the Formula I according to the process described above provides the corresponding hydrogenated compounds of Formula (IV):

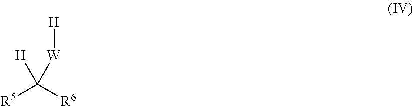

wherein W, R⁵ and R⁶ are defined as in Formula (III).

Since R⁵ and R⁶ may be different, it is hereby understood that the final product, of formula (IV), may be chiral, thus possibly consisting of a practically pure enantiomer or of a mixture of stereoisomers, depending on the nature of the catalyst used in the process.

The transfer hydrogenation conditions characterizing the process of the instant disclosure may comprise a base. Said base can be the substrate itself, if the latter is basic, or any conventional base. One can cite, as non-limiting examples, organic non-coordinating bases such as DBU, an alkaline or alkaline-earth metal carbonate, a carboxylate salt such as sodium or potassium acetate, or an alcoholate or hydroxide salt. The bases comprising alcoholate or hydroxide salts are selected from the group consisting of the compounds of formula (R¹⁰O)₂M′ and R¹⁰OM″, wherein M′ is an alkaline-earth metal, M″ is an alkaline metal and R¹⁰ stands for hydrogen or a linear or branched alkyl group.

Standard transfer hydrogenation conditions, as used herein, typically implies the mixture of the substrate with a compound of Formula I with a base, possibly in the presence of a solvent, and then treating such a mixture with a hydrogen donor solvent (such as isopropanol or a mixture of triethylamine and formic acid) at a chosen pressure and temperature. Varying the reaction conditions, including for example, temperature, pressure, solvent and reagent ratios, to optimize the yield of the desired product would be well within the abilities of a person skilled in the art.

The following non-limiting examples are illustrative of the present disclosure:

### EXAMPLES

The disclosure will now be described in further details by way of the following examples, wherein the temperatures are indicated in degrees centigrade and the abbreviations have the usual meaning in the art. All the procedures described hereafter have been carried out under an inert atmosphere unless stated otherwise. Where indicated, preparations and manipulations were carried out under H₂, N₂ or Ar atmospheres with the use of standard Schlenk, vacuum line and glove box techniques in dry, oxygen-free solvents. Tetrahydrofuran (THF), diethyl ether (Et₂O) and hexanes were purified and dried using an Innovative Technologies solvent purification system. Deuterated solvents were degassed and dried over activated molecular sieves. NMR spectra were recorded on a 300 MHz spectrometer (300 MHz for ¹H, 75 MHz for ¹³C and 121.5 for ³¹P). All ³¹P chemical shifts were measured relative to 85% H₃PO₄ as an external reference. ¹H and ¹³C chemical shifts were measured relative to partially deuterated solvent peaks but are reported relative to tetramethylsilane.

### Example 1

### Synthesis of Cationic Ruthenium Hydrogenation Catalysts

### (a) [Ru(p-cymene)(R,R-TsDPEN)]BF₄

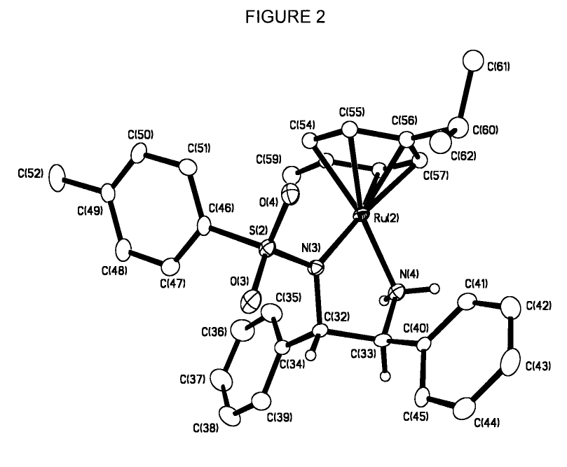

In an Ar filled flask, 0.150 g (0.24 mmol) of [RuCl(p-cymene)(R,R-TsDPEN)] and 0.046 g (0.24 mmol) of AgBF₄ were combined. CH₂Cl₂ (10 mL) was added and the resulting orange mixture was left to stir at ambient temperature. The orange suspension gradually darkened to brown and eventually to deep purple in colour. After 2 hours, the suspension was filtered through a 0.45 mm PTFE syringe filter. The purple filtrate was concentrated to dryness leaving a purple residue. Yield: 0.084 g (52%). ¹H NMR (ppm, CD₂Cl₂) showed the product was obtained. FIG. 2 shows the X-ray crystal structure of [Ru(p-cymene)(R,R-TsDPEN)]BF₄.

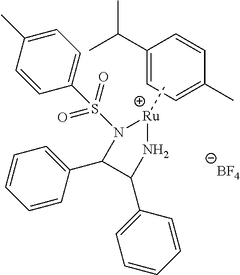

### (b) [Ru(p-cymene)(R,R-TsDPEN)]BF₄ (One-pot Procedure)

In an Ar filled flask, [RuCl₂(p-cymene)]₂ (0.100 g, 0.33 mmol Ru) was combined with AgBF₄ (0.064 g, 0.33 mmol) and (R,R)-TsDPEN (0.120 g, 0.33 mmol). Addition of THF (5 mL) resulted in a dark suspension which was stirred for 1-2 minutes and then NEt₃ (46 mL, 0.33 mmol) was added. The suspension was stirred at 80° C. for 1 hour then cooled to ambient temperature. The suspension was filtered through celite and the filtrate concentrated to dryness leaving a greenish brown residue. ¹H NMR (ppm, CD₂Cl₂) showed the product was obtained.

### (c) [Ru(p-cymene)(R,R-TsDPEN)]PF₆

In an Ar filled flask, 0.150 g (0.24 mmol) of [RuCl(p-cymene)(R,R-TsDPEN)] and 0.060 g (0.24 mmol) of AgPF₆ were combined. CH₂Cl₂ (10 mL) was added and the resulting orange mixture was left to stir at ambient temperature. The orange suspension gradually darkened to brown and eventually to deep purple in colour. After 21 hours, the suspension was filtered through a 0.45 mm PTFE syringe filter. The brown filtrate was concentrated to dryness leaving a brown residue. Yield: 0.150 g (85%). ¹H, ³¹P{¹H} and ¹⁹F{¹H} NMR (ppm, CDCl₃) showed the product was obtained.

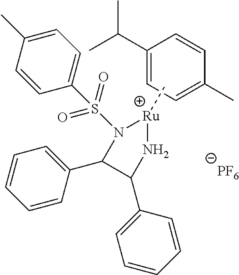

### (d) [Ru(p-cymene)(R,R-TsDPEN)]B(C₆F₅)₄

In an Ar filled flask, 0.100 g (0.16 mmol) of [RuCl(p-cymene)(R,R-TsDPEN)] and 0.205 g (0.24 mmol) of [Li(OEt₂)_{2.5}][B(C₆F₅)₄] were combined. CH₂Cl₂ (10 mL) was added and the resulting orange mixture was left to stir at ambient temperature overnight. 0.031 g of AgBF₄ was then added. The orange suspension gradually darkened to brown and eventually to deep purple in colour. After 21 hours, the suspension was filtered through a 0.45 mm PTFE syringe filter. The purple filtrate was concentrated to dryness leaving a purple residue. Yield: 0.175 g (87%). ¹H and ¹⁹F{¹H} NMR (ppm, CDCl₃) showed the product was obtained.

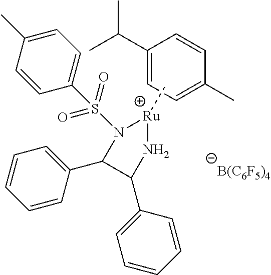

### (e) [Ru(p-cymene)(R,R-TsDPEN)]B[3,5-(CF₃)₂C₆H₃)]₄

In an Ar filled flask, 0.08 g (0.13 mmol) of [RuCl(p-cymene)(R,R-TsDPEN)] and 0.111 g (0.13 mmol) of NaB[3,5-(CF₃)₂C₆H₃)]₄ and 0.025 g (0.13 mmol) of AgBF₄ were combined. CH₂Cl₂ (5 mL) was added and the resulting brown purple mixture was left to stir at ambient temperature overnight. After 21 hours, the suspension was filtered through a 0.45 mm PTFE syringe filter. The purple filtrate was concentrated to dryness leaving a purple residue. Yield: 0.145 g (70%). ¹H and ¹⁹F{¹H} NMR (ppm, CDCl₃) showed the product was obtained.

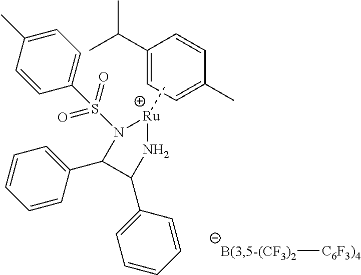

### (f) [Ru(p-cymene)(R,R-TsDPEN)]OTf and [Ru(p-cymene)(R,R-TsDPEN)(OTf)]

In an Ar filled flask, 0.150 g (0.24 mmol) of [RuCl(p-cymene)(R,R-TsDPEN)] and 0.037 g (0.24 mmol) of LiOTf were combined. CH₂Cl₂ (10 mL) was added and the resulting orange mixture was left to stir at ambient temperature. 0.046 g of AgBF₄ was then added. The orange suspension gradually darkened to brown and eventually to deep purple in colour. After 21 hours, the suspension was filtered through a 0.45 mm PTFE syringe filter. The brown filtrate was concentrated to dryness leaving a brown residue. Yield: 0.120 g (68%). ¹H and ¹⁹F{¹H} NMR (ppm, CD₂Cl₂) showed that the product was obtained (ratio of the products: 1:1).

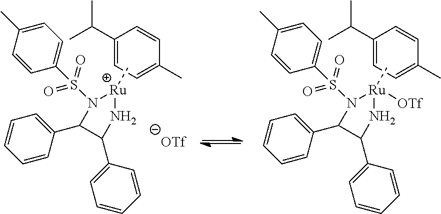

### (g) [Ru(p-cymene)(R,R-TsDPEN)(pyridine)]BF₄

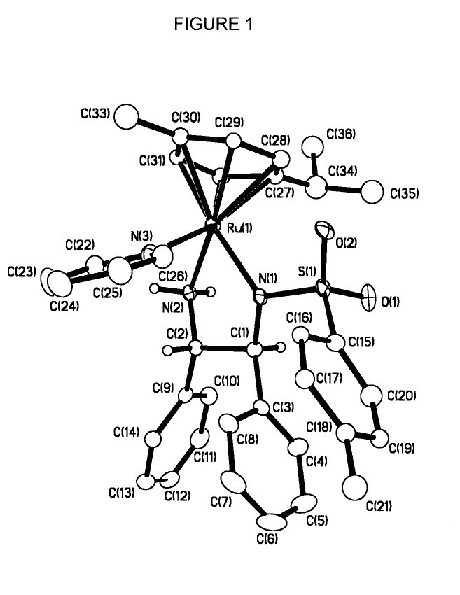

To a solution of 0.015 g (0.022 mmol) of [Ru(p-cymene)(R,R-TsDPEN)]BF₄ in 1 mL of CD₂Cl₂ was added 1.8 mL of pyridine. The originally purple solution instantly turned golden yellow in colour. ¹H NMR analysis showed the product was obtained. FIG. 1 shows the X-ray crystal structure of [Ru(p-cymene)(R,R-TsDPEN)(pyridine)]BF₄.

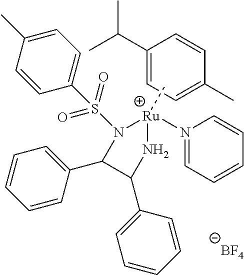

### (h) [Ru(p-cymene)(R,R-TsDPEN)(pyridine)]PF₆

In an Ar filled flask, 0.130 g (0.2 mmol) of [RuCl(p-cymene)(R,R-TsDPEN)] and 0.052 g (0.2 mmol) of AgPF₆ were combined. CH₂Cl₂ (10 mL) was added and the resulting orange mixture was left to stir at ambient temperature overnight. The orange suspension gradually darkened to brown and eventually to deep purple in colour. After 16 hours, the suspension was filtered through a 0.45 mm PTFE syringe filter and 16 mL of pyridine (0.2 mmol) was added. The resulting yellow solution was concentrated to dryness leaving a yellow residue. Yield: 0.150 g (89%). ¹H NMR, ³¹P{¹H} NMR and ¹⁹F{¹H} NMR (ppm, CDCl₃) showed the product was obtained.

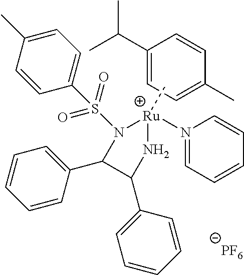

### (i) [Ru(p-cymene)(R,R-TsDPEN)(pyridine)]OTf

In an Ar filled flask, 0.130 g (0.2 mmol) of [RuCl(p-cymene)(R,R-TsDPEN)] and 0.053 g (0.2 mmol) of AgOTf were combined. CH₂Cl₂ (10 mL) was added and the resulting orange mixture was left to stir at ambient temperature overnight. The orange suspension gradually darkened to brown and eventually to deep purple in colour. After 16 hours, the suspension was filtered through a 0.45 mm PTFE syringe filter and 16 mL (0.2 mmol) of pyridine was added. The resulting yellow solution was concentrated to dryness leaving a yellow residue. Yield: 0.130 g (76%). ¹H NMR and ¹⁹F{¹H} NMR (ppm, CDCl₃) showed the product was obtained.

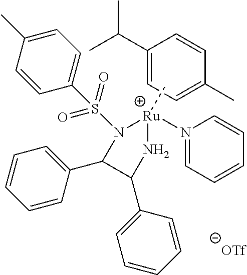

### (j) [Ru(p-cymene)(R,R-TsDPEN)(pyridine)]B(C₆F₅)₄

In an Ar filled flask, 0.082 g (0.13 mmol) of [RuCl(p-cymene)(R,R-TsDPEN)], 0.112 g (0.24 mmol) of LiB(C₆F₅)₄ and 0.025 g of AgBF₄ were combined. CH₂Cl₂ (10 mL) was added and the resulting orange mixture was left to stir at ambient temperature overnight. The orange suspension gradually darkened to brown and eventually to deep purple in colour. After 16 hours, the suspension was filtered through a 0.45 mm PTFE syringe filter and 10 mL (0.13 mmol) of pyridine was added. The yellow solution was concentrated to dryness leaving a purple residue. Yield: 0.160 g (92%). ¹H NMR and ¹⁹F{¹H} NMR (ppm, CDCl₃) showed the product was obtained.

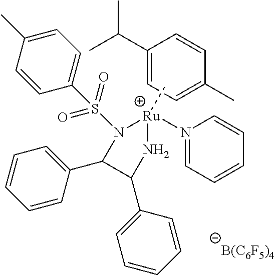

### (k) Ru(p-cymene)(R,R-TsDPEN)(pyridine)]B[3,5-(CF₃)₂C₆H₃)]₄

In an Ar filled flask, 0.080 g (0.13 mmol) of [RuCl(p-cymene)(R,R-TsDPEN)], 0.114 g (0.13 mmol) of NaB[3,5-(CF₃)₂C₆H₃)]₄ and 0.025 g of AgBF₄ (0.13 mmol) were combined. CH₂Cl₂ (5 mL) was added and the resulting brown-purple mixture was left to stir at ambient temperature overnight. After 16 hours, the suspension was filtered through a 0.45 mm PTFE syringe filter and 10 mL (0.13 mmol) of pyridine was added. The resulting brown solution was concentrated to dryness leaving a brown residue. Yield: 0.150 g (72%). ¹H NMR and ¹⁹F{¹H} NMR (ppm, CDCl₃) showed the product was obtained.

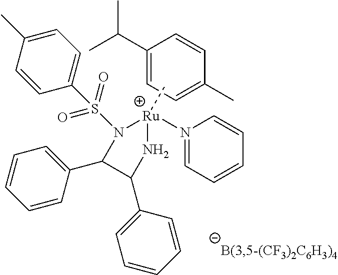

### Example 2

### Transfer Hydrogenation of Acetophenone

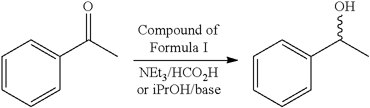

### (a) General Procedure for Transfer Hydrogenation of Acetophenone in 2-Propanol (IPA)

To a solution of acetophenone (1.00 g, 8.32 mmol) in 5 mL of 2-propanol was added 2.0 mL of a 0.1 M solution of KOH in 2-propanol. To this solution was added the solid catalyst (0.011 g, 0.017 mmol). The mixture was stirred at the desired temperature for the specified time. The sample was then filtered through silica gel (ca. 2 cm) using CH₂Cl₂ and submitted for GC analysis. For experiments without KOH, 2.0 mL of 2-propanol was used to maintain equivalent concentrations of the remaining reagents. Results are shown in Table 1.

### Discussion

The four different anions studied (i.e. BF₄, PF₆, B(C₆F₅)₄, and OTf) all showed similar enantioselectivities at 25° C. (see entries 2, 4, 8 and 12 of Table 1) between 94-96% e.e. In terms of activity, the PF₆ complex gave the highest conversion of 49% while the OTf complex gave the lowest conversion of 23%. It should be noted that these are unoptimized conditions. At higher temperatures (i.e. 80° C.) the enantioselectivities dropped (on the order of 20% for each complex) while the conversions showed more disparate behaviour. In the case of PF₆, the conversion was unchanged (see entry 4 vs. 7) however for both B(C₆F₅)₄ and OTf the conversions significantly improved at elevated temperatures (see entry 8 vs. 11 and 12 vs. 15 respectively). The requirement for added base in this solvent system (in this case KOH) was also examined for the BF₄ complex. It was found that with no added base there is no activity in the transfer hydrogenation of acetophenone (see entry 1 vs. 2).

When compared to the chloride complex under these conditions (i.e. in 2-propanol solvent) the cationic complexes generally give lower conversions but comparable enantioselectivities. The closest competitor among the cations is the B(C₆F₅)₄ complex. After 2 hours, this cation gives a conversion of 73% with an e.e. of 80% compared to 84% conversion and 90% e.e. for the neutral chloride. It should be noted that the conditions employed are unoptimized. Examination of the data for the B(C₆F₅)₄ cation (compare Entries 9-11) clearly shows that a maximum for both conversion and e.e. is reached somewhere between the 1 and 20 h time frame but is not represented by the data available. A similar conclusion emerges for the PF₆ cation (Entries 5-7).

### (b) General Procedure for Transfer Hydrogenation of acetophenone in NEt₃/Formic Acid

To 2 mL of a previously prepared mixture of formic acid/NEt₃ (1.5:1) was added acetophenone (1.00 g, 8.32 mmol). To this solution was added the solid catalyst (0.011 g, 0.017 mmol). The mixture was stirred at the specified temperature for the specified time. The sample was then filtered through silica gel (ca. 2 cm) using CH₂Cl₂ and submitted for GC analysis. For experiments involving no added NEt₃, 2.0 mL of formic acid was used to maintain equivalent concentrations of the remaining reagents. Results are shown in Table 2.

### Discussion

In this solvent system, enantioselectivities were high for all catalysts tested and conversions were also generally high. As with 2-propanol, the B(C₆F₅)₄, and OTf complexes gave lower conversions than the BF₄ and PF₆ complexes (see entries 4, 5, 2 and 3 respectively). Similar to the isopropanol solvent system, at elevated temperatures (for the OTf complex) enantioselectivity dropped while conversion increased (see entry 5 vs. 7).

A complex derived from a chiral counterion, (BINO)₂B (see entry 8), was also tested. The enantioselectivity matched that of the complexes incorporating the achiral anions however the conversion was quite low. The low conversion is ascribed to the fact that the sample was obtained from an NMR sample solution thus purity is questionable. The high enantioselectivity was not surprising as the chiral Ru cations are expected to dominate enantioselection. Testing with achiral Ru cations is currently underway to determine if the chiral counterion alone can affect enantioselectivity.

A sample of the BF₄ cation prepared in a ‘one-pot’ procedure (i.e. starting from [RuCl₂(p-cymene)]₂ and without isolating the intermediate, [RuCl(p-cymene)(R,R-TsDPEN)]) was also tested (see entry 9). The enantioselectivity and the conversion matched that of the material prepared from the isolated precursor (see entry 2 vs. 10). This represents an alternative synthetic route, direct to the cation, foregoing the need to isolate the chloride complex.

The comparison between the cations and the neutral chloride complex in this solvent system reveals the cationic complexes to be at least as good as the precursor complex (see entries 3-5 to 10). In fact, all of the cations give slightly higher enantioselectivity (97% for the cations compared to 96% for the chloride). The PF₆ cation matches the chloride in conversion while the remaining cations display slightly lower conversions.

Lewis base adducts of the cations are also readily prepared by adding the desired base to the cation (or by exposing the cation to the base during the synthesis). The resulting complexes are generally highly air- and moisture-stable complexes (even more so than the corresponding base-free compounds) and isolated in high yield and purity. These complexes are also effective catalysts in the transfer hydrogenation of ketones. The pyridine adducts of the cations described above have been prepared and tested in analogy to the base-free precursors (see table 3). The enantioselectivity is not affected by coordinated pyridine (compare Table 2 Entries 3, 5 and 6 with Table 3 Entries 2, 3 and 4 respectively), however conversion seems to improve for the B(C₆F₅)₄ and OTf complexes upon coordination of pyridine. This is likely due to the improved stability, particularly at the slightly elevated temperatures of the catalysis, of the pyridine adducts relative to the ‘naked’ base-free cations. The presence of the vacant site in the base-free cations makes those complexes more susceptible to degradation (relative to the coordinatively saturated Lewis base adducts) resulting in reduced conversions.

### Example 3

### Transfer Hydrogenation of 2,3,3-trimethylindolenine

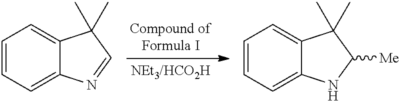

### (a) General Procedure for Transfer Hydrogenation of 2,3,3-trimethylindolenine in NEt₃/Formic Acid

To 2 mL of a previously prepared mixture of formic acid/NEt₃ (1.5:1) was added 2,3,3-trimethylindolenine (55 mg, 0.35 mmol). To this solution was added the solid catalyst (0.004 g, 0.007 mmol). The mixture was stirred at the specified temperature for the specified time. A solution of Na₂CO₃ was added to render the mixture basic. The product was extracted with CH₂Cl₂. The resulting organic phases were dried using MgSO₄, filtered and evaporated to dryness. The sample was then filtered through silica gel (ca. 2 cm) using CH₂Cl₂ and submitted for HPLC analysis to determine the e.e. The ¹H NMR analysis was used to calculate the conversion. Results are shown in Table 4.

### Example 4a

### Transfer Hydrogenation of a Range of Ketone and Imine Substrates using TEAF(triethylamine/formic acid)

A test tube equipped with a stir bar was charged with substrate (500 eq) and catalyst (1 eq). To this was added 2 mL of a solution of formic acid and triethylamine (3:2 equivalence) and 1 mL of dichloromethane. The resulting solution was stirred at 40° C. for 18 h. The solution was then transferred to a round-bottom flask using dichloromethane. If suitable for GC analysis, the solution was filtered through silica gel using EtOAc as eluent, and injected into the GC apparatus for determination of % Conversion and ee. For HPLC analysis, the solvent was removed under reduced pressure to yield an oil. The oil was redissolved in dichloromethane and purified using silica gel chromatography (30% EtOAc in hexane). The conversion was determined by ¹H NMR spectroscopy (CDCl₃) and the ee determined by HPLC. The results are shown in Table 5.

### Example 4b

### Transfer Hydrogenation of a Range of Ketone and Imine Substrates using IPA (isopropyl alcohol)

A test tube equipped with a stir bar was charged with a solution of catalyst 1 ([Ru(p-cymene)(S,S-TsDPEN)]BF₄, 0.00873 mmol, 1 eq) and acetophenone (4.37 mmol, 500 eq) or of catalyst 2 ([Ru(p-cymene)(S,S-TsDPEN)(pyridine)]BF₄, 0.00848 mmol, 1 eq) and acetophenone (4.24 mmol, 500 eq). To this was added 2.5 mL isopropanol and 1 mL of KOH/isopropanol solution (1 eq, 5 eq, 10 eq, 50 eq). The resulting solution was stirred at 40° C. for 20 h under Ar. The solution was then filtered through silica gel using CH₂Cl₂ as eluent, and a sample injected into the GC apparatus for determination of % Conversion and ee.

### Example 5a

### Effect of Time and Base in Transfer Hydrogenation of Acetophenone

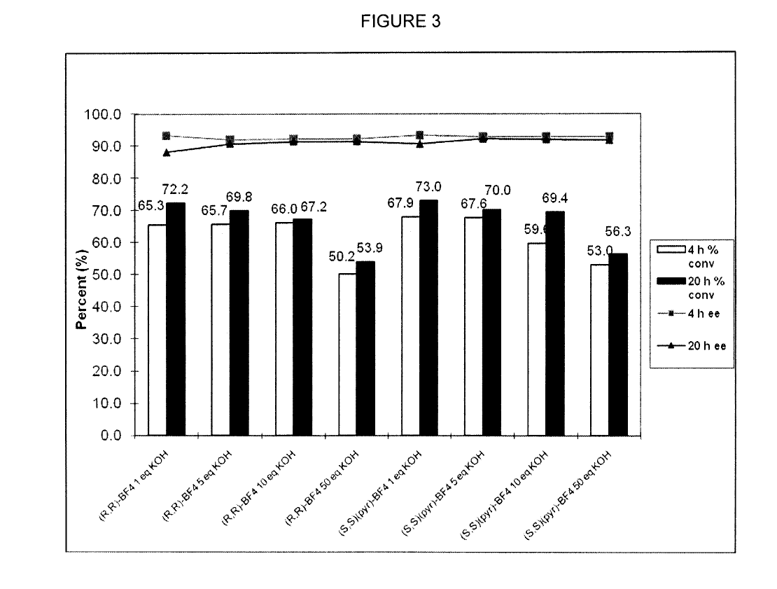

The same experimental procedure as Example 4b was used as to determine the conversion and enantiomeric excess values for the transfer hydrogenation of acetophenone. The results are shown in FIG. 3.

### Example 5b

### Effect of Triethylamine/Formic Acid Volume and Co-Solvent in Transfer Hydrogenation of Acetophenone

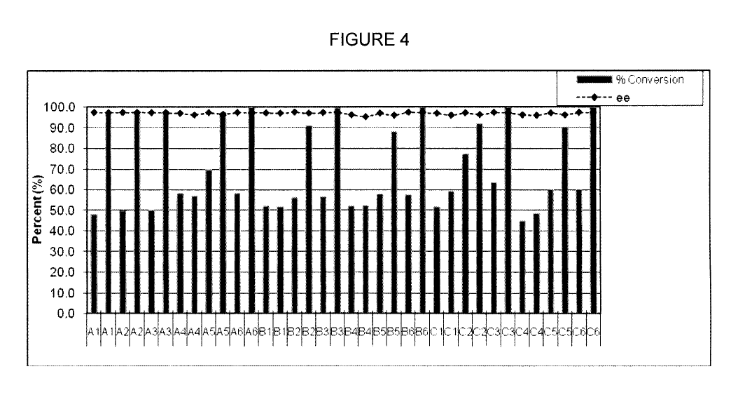

The same experimental procedure as Example 4a was used as to determine the conversion and enantiomeric excess values for the transfer hydrogenation of acetophenone. The results are shown in FIG. 4.

### Example 5c

### Effect of Triethylamine/Formic Acid Volume, Co-Solvent and Water in Transfer Hydrogenation of Acetophenone

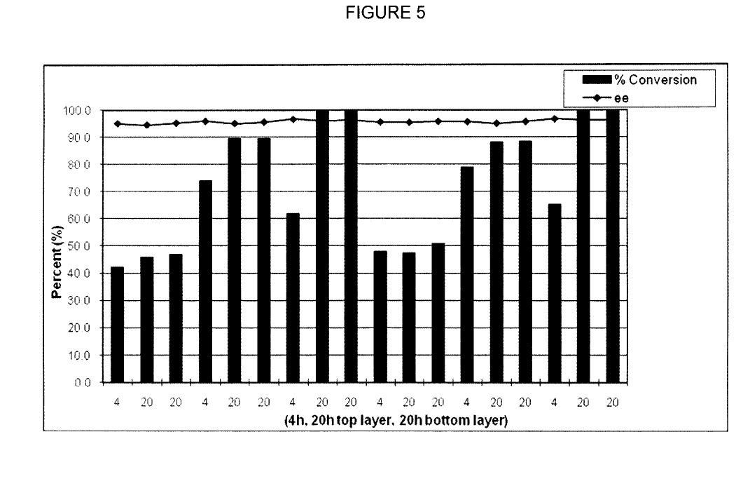

The same experimental procedure as Example 4a was used as to determine the conversion and enantiomeric excess values for the transfer hydrogenation of acetophenone. The results are shown in FIG. 5.

The results shown in FIGS. 4 and 5 are interpreted using the chart below:

| Expt ID | 1 | 2 | 3 | 4 | 5 | 6 |
|---|---|---|---|---|---|---|
| A | 2.0 mL | 2.0 mL | 2.0 mL | 0.5 mL | 1.0 mL | 1.5 mL |
| | HCOOH/NEt₃ | HCOOH/NEt₃ | HCOOH/NEt₃ | HCOOH/NEt₃ | HCOOH/NEt₃ | HCOOH/NEt₃ |
| B | 0.5 mL | 1.0 mL | 1.5 mL | 0.5 mL | 1.0 mL | 1.5 mL |
| | HCOOH/NEt₃ | HCOOH/NEt₃ | HCOOH/NEt₃ | HCOOH/NEt₃ | HCOOH/NEt₃ | HCOOH/NEt₃ |
| | 1.5 mL THF | 1.0 mL THF | 0.5 mL THF | 1.5 mL CH₂Cl₂ | 1.0 mL CH₂Cl₂ | 0.5 mL CH₂Cl₂ |
| C | 0.5 mL | 1.0 mL | 1.5 mL | 0.5 mL | 1.0 mL | 1.5 mL |
| | HCOOH/NEt₃ | HCOOH/NEt₃ | HCOOH/NEt₃ | HCOOH/NEt₃ | HCOOH/NEt₃ | HCOOH/NEt₃ |
| | 1.5 mL Et₂O | 1.0 mL Et₂O | 0.5 mL Et₂O | 1.5 mL i-PrOH | 1.0 mL i-PrOH | 0.5 mL i-PrOH |
| D | 0.5 mL | 1.0 mL | 1.5 mL | 0.5 mL | 1.0 mL | 1.5 mL |
| | HCOOH/NEt₃ | HCOOH/NEt₃ | HCOOH/NEt₃ | HCOOH/NEt₃ | HCOOH/NEt₃ | HCOOH/NEt₃ |
| | 0.75 mL CH₂Cl₂ | 0.5 mL CH₂Cl₂ | 0.25 mL CH₂Cl₂ | 0.75 mL Et₂O | 0.5 mL Et₂O | 0.25 mL Et₂O |
| | 0.75 mL H₂O | 0.5 mL H₂O | 0.25 mL H₂O | 0.75 mL H₂O | 0.5 mL H₂O | 0.25 mL H₂O |

### Example 6a

### N-](1R,2R)-1,2-diphenyl 2-3-(3-phenylpropylamino)-ethyl]-4-methylbenzenesulfonamide ruthenium(II) tetrafluoroborate

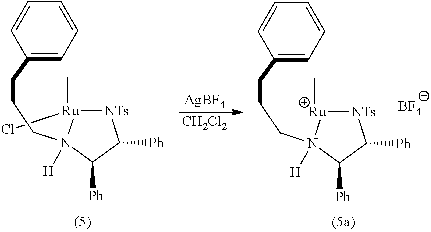

In an Ar filled flask, 25 mg (0.04 mmol) of N-[(1R,2R)-1,2-diphenyl 2-3-(3-phenylpropylamino)-ethyl]-4-methylbenzenesulfonamide chloro ruthenium(II) and 8 mg (0.04 mmol) of AgBF₄ were combined. CH₂Cl₂ (2 mL) was added and the resulting orange coloured suspension was left to stir at ambient temperature for 5 hours after which time it was filtered through Celite. The orange filtrate was reduced to dryness leaving an orange residue. Yield: 27 mg (quantitative yield). ¹H NMR (ppm, CDCl₃): 1.25 (s), 1.43-1.47 (m), 1.83-1.97 (m), 2.18-2.36 (m), 2.66-2.71(m), 2.82-2.95 (m), 3.73-3.77 (m), 3.87-4.33 (m), 5.23-5.30, 5.59-5.64 (m), 5.91-5.96 (m), 6.44-7.46 (m). ¹⁹F NMR (ppm, CDCl₃): −150 (s).

### 6b: N-[(1R,2R)-1,2-diphenyl 2-3-(3-phenylpropylamino)-ethyl]-4-methylbenzene-sulfonamide(pyridine)ruthenium(II) tetrafluoroborate

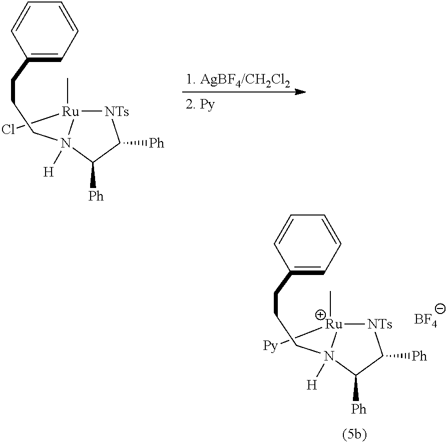

In an Ar filled flask, 10 mg (0.016 mmol) of N-[(1R,2R)-1,2-diphenyl 2-3-(3-phenylpropylamino)-ethyl]-4-methylbenzenesulfonamide chloro ruthenium(II) and 3 mg (0.0016 mmol) of AgBF₄ were combined. CH₂Cl₂ (2 mL) was added and the resulting orange coloured suspension was left to stir at ambient temperature for 2.5 hours after which time it was filtered through Celite. Py (1.3 mL, 0.016 mmol) was added. The yellow solution was reduced to dryness leaving an orange residue. Yield: 12 mg (quantitative yield). ¹H NMR (ppm, CDCl₃): 1.83-1.89 (m), 2.17-2.35 (m), 2.87-3.17 (m) (m), 3.60 (d, J=11 Hz), 3.72-3.76 (m), 4.73 (d, J=12 Hz), 5.37 (t, J=6 Hz), 5.81-5.87 (m), 6.01-6.03 (m), 6.46-7.77 (m), 8.02-8.04 (m), 8.64 (s), 9.41-9.47 (m). ¹⁹F NMR (ppm, CDCl₃): −150 (s).

### Example 7

### General Procedure for Transfer Hydrogenation in NEt₃/Formic Acid Using Tethered Catalysts

The catalyst (5, 5a or 5b) (0.011 g, 0.016 mmol) was dissolved in acetophenone (1.00 g, 8.32 mmol). 1 mL of a previously prepared mixture of formic acid/NEt₃ (1.5:1) was added to this solution. The mixture was stirred at 40° C. The sample was then filtered through silica gel (ca. 2 cm) using CH₂Cl₂ and submitted for GC analysis. The results are shown in Table 6.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
2-Propanol Transfer Hydrogenation of Acetophenone
Catalyzed by [Ru(p-cymene)(R,R-TsDPEN)]X.

| | | Temp | Time | Conv. | e.e. |
|---|---|---|---|---|---|
| Entry | X | (° C.) | (h) | (%) | (%) |
| 1^{a} | BF₄ | 25 | 16 | 0 | — |
| 2 | BF₄ | 25 | 16 | 43 | 94 |
| 3 | PF₆ | 25 | 1.5 | 13 | 96 |
| 4 | PF₆ | 25 | 16 | 49 | 95 |
| 5 | PF₆ | 80 | 1 | 49 | 85 |
| 6 | PF₆ | 80 | 2 | 53 | 83 |
| 7 | PF₆ | 80 | 19.7 | 49 | 77 |
| 8 | B(C₆F₅)₄ | 25 | 16 | 37 | 95 |
| 9 | B(C₆F₅)₄ | 80 | 1 | 62 | 84 |
| 10 | B(C₆F₅)4 | 80 | 2 | 73 | 80 |
| 11 | B(C₆F₅)4 | 80 | 19.7 | 57 | 63 |
| 12 | OTf | 25 | 16 | 23 | 96 |
| 13 | OTf | 80 | 1 | 46 | 85 |
| 14 | OTf | 80 | 2 | 49 | 83 |
| 15 | OTf | 80 | 19.7 | 56 | 72 |
| 16^{b} | Cl | 80 | 1 | 26 | 94 |
| 17^{b} | Cl | 80 | 18 | 84 | 90 |
| 18 | B(3,5-(CF₃)₂C₆H₃)₄ | 25 | 17 | 42 | 94 |
| 19 | B[3,5-(CF₃)₂C₆H₃)₄ | 25 | 17 | 54 | 94 |

^{a}No base added.
^{b}Corresponds to chloride compound RuCl(p-cymene)(TsDPEN) (i.e. starting material for the derived cations)

TABLE 2
NEt₃/Formic Acid Transfer Hydrogenation of Acetophenone
Catalyzed by [Ru(p-cymene)(R,R-TsDPEN)]X.

| | | Temp | Time | Conv. | e.e. |
|---|---|---|---|---|---|
| Entry | X | (° C.) | (h) | (%) | (%) |
| 1^{a} | BF₄ | 40 | 16 | 0 | — |
| 2 | BF₄ | 40 | 16 | 94 | 96 |
| 3 | PF₆ | 40 | 16.7 | >99 | 97 |
| 4 | B(C₆F₅)₄ | 40 | 16.7 | 84 | 97 |
| 5 | OTf | 40 | 16.7 | 75 | 97 |
| 6 | OTf | 80 | 1.3 | 89 | 94 |
| 7 | OTf | 80 | 18.7 | 90 | 93 |
| 8^{b} | (R—BINO)₂B | 40 | 18.5 | 20 | 95 |
| 9^{c} | BF₄ | 40 | 19 | 93 | 97 |
| 10^{d} | Cl | 40 | 18 | 99 | 96 |
| 11 | B(3,5-(CF₃)₂C₆H₃)₄ | 40 | 17 | 99 | 96 |

^{a}No NEt₃ added.
^{b}Sample generated from an NMR tube synthesis of catalyst: 10 mg of RuCl(p-cymene)(TsDPEN) and 10 mg of Ag[(BINO)₂B] combined and 1 mL CD₂Cl₂ added. Resulting suspension stirred briefly then filtered through 0.45 μm PTFE syringe filter and filtrate collected and analyzed by NMR. Sample retrieved and used for testing based on reagent concentrations and assumed complete conversion to desired product.
^{c}Catalyst prepared in a one-pot procedure; see experimental for details.
^{d}Corresponds to chloride compound RuCl(p-cymene)(TsDPEN) (i.e. starting material for the derived cations)

TABLE 3
NEt₃/Formic Acid Transfer Hydrogenation of Acetophenone
Catalyzed by [Ru(p-cymene)(R,R-TsDPEN)(pyridine)]X.

| | | Temp | Time | Conv. | e.e. |
|---|---|---|---|---|---|
| Entry | X | (° C.) | (h) | (%) | (%) |
| 1 | BF₄ | 40 | 16 | | |
| 2 | PF₆ | 40 | 20.5 | 95 | 97 |
| 3 | B(C₆F₅)₄ | 40 | 20.5 | 100 | 97 |
| 4 | OTf | 40 | 20.5 | 94 | 97 |
| 5 | B(3,5- | 40 | 17 | 93 | 96 |
| | (CF₃)₂C₆H₃)4 | | | | |

TABLE 4
NEt₃/Formic Acid Transfer Hydrogenation of 2,3,3-trimethylindolenine
Catalyzed by [Ru(p-cymene)(R,R-TsDPEN)]X.

| | | Temp | Time | Conv. | e.e. |
|---|---|---|---|---|---|
| Entry | X | (° C.) | (h) | (%)^{a} | (%)^{b} |
| 1 | PF₆ | 40 | 19 | 33 | 20 |
| 2 | B(3,5-(CF₃)₂C₆H₃)₄ | 40 | 19 | 36 | 25 |
| 3^{c,d} | Cl | 40 | 21 | 63 | 41 |
| 4^{c,e} | Cl | 40 | 19 | 23 | 41 |

^{a}Determined by ¹H NMR.
^{b}Determined by HPLC.
^{c}Corresponds to chloride compound RuCl(p-cymene)(TsDPEN) (i.e. starting
material for the derived cations).
^{d}There are others signals in the ¹H NMR which remain unassigned.
^{e}To 2 mL of a previously prepared mixture of formic acid/NEt₃ (2.5:1) was added 2,3,3-trimethylindolenine (55 mg, 0.35 mmol) and 5 mL of MeCN. To this solution was added the solid catalyst (0.004 g, 0.007 mmol).

TABLE 5
Transfer Hydrogenation of A Range of Ketone and Imine
Substrates.

| | 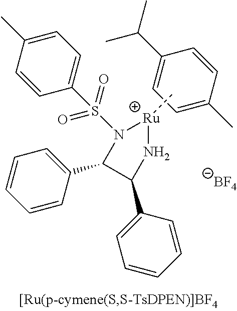 | | 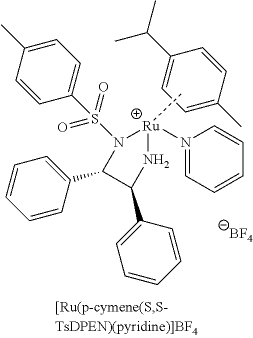 | |
|---|---|---|---|---|
| Catalyst Substrate | % Conversion | ee | % Conversion | ee |
| 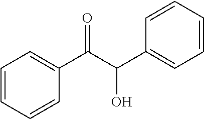 | 80.4 | 52.6 | 90.5 | 91.8 |
| 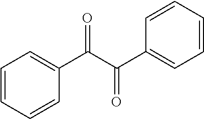 | 90.1 | 98.5 | 95.2 | 97.7 |
| 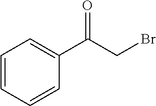 | 97.4 | 86.8 | 99.2 | 87.8 |
| 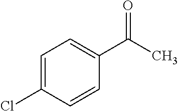 | 99.8² | 92.6² | 99.7² | 92.6² |
| 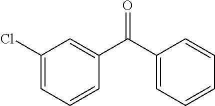 | >99 | 23.3 | >99 | 30.9 |
| 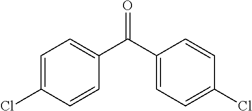 | 100 | 0 | >99 | 0 |
| 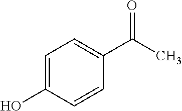 | 34.4 | 96.1 | 40.8 | 94.6 |
| 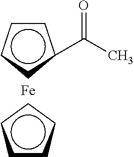 | 20.5 | >99 | 26.0 | >99 |
| 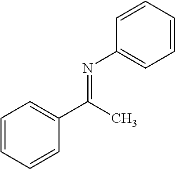 | 58.7 | 94.7 | 35.5 | 95.7 |
| 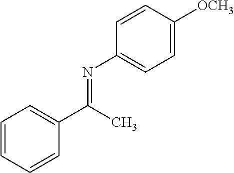 | 37.5 | 70.9 | 68.3 | 46.8 |

¹Conversion by ¹H NMR spectroscopy and ee by HPLC unless otherwise specified.
²Conversion and ee by GC.

TABLE 6
NEt₃/Formic Acid Transfer Hydrogenation Results

| | | Temp | Time | Conv. | e.e. |
|---|---|---|---|---|---|
| Entry | Catalyst | (° C.) | (h) | (%) | (%) |
| 1 | 5 | 40 | 19 | 83 | 89(R) |
| 2 | 5a | 40 | 17 | >99 | 96(K) |
| 3 | 5b | 40 | 18 | >99 | 96(R) |

### FULL CITATIONS FOR DOCUMENTS REFERRED TO IN THE SPECIFICATION

1. Noyori et al., J. Am. Chem. Soc. 1995, 117, 7562.
2. Noyori et al., J. Am. Chem. Soc. 1996, 118, 4916.
3. See for example: WO2006137167, WO2006137165, WO2006137195, WO2002051781, JP10236986 and WO9720789.
4. (a) Ikariya et al., J. Am. Chem. Soc. 2003, 125, 7508. (b) Ikariya et al., J. Am. Chem. Soc. 2004, 126, 11148.
5. Ikariya et al., Tetrahedron Lett. 2005, 46, 963.
6. Kobayashi, S. WO 2003076478 A1.
7. (a) Hannedouche, J.; Clarkson, G. J.; Wills, M. J. Am. Chem. Soc. 2004, 126, 986. (b) Hayes, A. M.; Morris, D. J.; Clarkson, G. J.; Wills, M. J. Am. Chem. Soc. 2005, 127, 7318.

1. A compound of Formula I:
[Ru(D-Z¹—NHR¹)(Ar)(LB)ₙ]^{r+}[Y⁻]ᵣ (I)
wherein
Ar is optionally substituted aryl, wherein the optional substituents are selected from one or more of halo, C₁₋₆alkyl, fluoro-substituted-C₁₋₆alkyl, C₂₋₆alkenyl, C₂₋₆alkynyl, aryl and fluoro-substituted aryl, and Ar is optionally linked to a polymeric support;
LB is any neutral Lewis base;
Y is any non-coordinating anion;
n is 0 or 1;
r is 1 or 2;
D-Z¹−NHR¹ is a coordinated bidentate ligand in which
Z¹ is C₂-C₇alkylene, C₄-C₁₀cycloalkylene, metallocenediyl, C₆-C₂₂arylene or combinations of one or more of C₂-C₇alkylene, C₄-C₁₀cycloalkylene, metallocenediyl and C₆-C₂₂arylene, said C₂-C₇alkylene, C₄-C₁₀cycloalkylene, metallocenediyl and C₆-C₂₂arylene groups being optionally substituted, wherein the optional substituents are selected from one or more of halo, C₁₋₆alkyl, fluoro-substituted-C₁₋₆alkyl, C₂₋₆alkenyl, C₂₋₆alkynyl, aryl and fluoro-substituted aryl;
D is NR², OR², SR², SeR² or TeR²;
R² is H, C₁₋₂₀alkyl, S(O)₂R³, P(O)(R³)₂, C(O)R³, C(O)N(R³)₂ or C(S)N(R³)₂; and
R¹ and R³, are simultaneously or independently H, C₁₋₈alkyl, C₂₋₈alkenyl, C₃₋₁₀cycloalkyl or aryl, said latter 4 groups being optionally substituted wherein the optional substituents are selected from one or more of halo, C₁₋₆alkyl, fluoro-substituted-C₁₋₆alkyl, C₂₋₆alkenyl, C₂₋₆alkynyl, aryl and fluoro-substituted aryl,
or R¹ and Ar, or R² and Ar, are linked via Z²,
wherein Z² is as defined as Z¹ above, and wherein one or more carbon atoms in Z² is optionally replaced with —O—, —S—, —C(═O)—, —S(═O)—, —S(═O)₂—, —PR³—, —P(═O)R³—, NH or NR³.

2. The compound according to claim 1, wherein Ar is optionally substituted phenyl, the optional substituents being selected from one or more of halo, C₁₋₆alkyl fluoro-substituted-C₁₋₆alkyl, C₂₋₆alkenyl, C₂₋₆alkynyl, aryl and fluoro-substituted aryl.

3. The compound according to claim 2, wherein Ar is

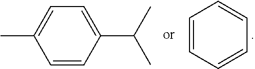

4. The compound according to claim 1, wherein Ar is linked to a polymeric support.

5. (canceled)

6. The compound according to claim 1, wherein Z¹ is optionally substituted C₂-C₄alkylene, C₅₋₈cycloalkylene, ferrocendiyl, phenylene, naphthylene or bisphenylene, wherein the optional substituents are selected from one or more of halo, C₁₋₄alkyl, fluoro-substituted-C₁₋₄alkyl, phenyl and fluoro-substituted phenyl.

7. The compound according to claim 6, wherein Z¹ is optionally substituted C₂₋₄alkylene wherein the optional substituents are selected from one or two of halo, C₁₋₄alkyl, fluoro-substituted-C₁₋₄alkyl, phenyl and fluoro-substituted phenyl.

8. The compound according to claim 1, wherein D is NR².

9. (canceled)

10. The compound according to claim 1, wherein R¹ and R³, are simultaneously or independently, H, C₁₋₆alkyl, C₂₋₆alkenyl, C₅₋₈cycloalkyl or aryl, said latter 4 groups being optionally substituted wherein the optional substituents are selected from one or more of halo, C₁₋₄alkyl, fluoro-substituted-C₁₋₄alkyl, phenyl and fluoro-substituted phenyl.

11. The compound according to claim 10, wherein R¹ and R³ are simultaneously or independently, H, C₁₋₆alkyl, C₂₋₆alkenyl, C₅₋₈cycloalkyl or phenyl, said latter four groups being optionally substituted wherein the optional substituents are selected from one or more of halo, C₁₋₄alkyl, fluoro-substituted-C₁₋₄alkyl, phenyl and fluoro-substituted phenyl.

12. The compound according to claim 11, wherein R³ is

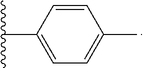

13. The compound according to claim 11, wherein R¹ is H.

14. The compound according to claim 1 wherein D-Z¹—NHR¹ is chiral.

15. The compound according to claim 14, wherein D-Z¹—NHR¹ is selected from those in which NHR¹ is stereogenic and those in which both D and NHR¹ are chiral.

16. (canceled)

17. The compound according to claim 1, wherein Y is OTf, BF₄, PF₆, B(C₁₋₆alkyl)₄, B(fluoro-substituted-C₁₋₆alkyl)₄, B(aryl)₄ wherein aryl is unsubstituted or substituted 1-5 times with fluoro, C₁₋₄alkyl or fluoro-substituted C₁₋₄alkyl, or

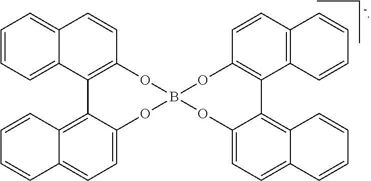

18. The compound according to claim 1, wherein Z² is C₂-C₄alkylene, C₅₋₈cycloalkylene, ferrocendiyl, phenylene, naphthylene or bisphenylene, said 6 group being optionally substituted wherein the optional substituents are selected from one or more of halo, C₁₋₄alkyl, fluoro-substituted-C₁₋₄alkyl, phenyl and fluoro-substituted phenyl and wherein one or more carbon atoms in Z² is optionally replaced with —O—, —S—, —C(═O)—, —S(═O)—, —S(═O)₂—, —PR³—, —P(═O)R³—, NH or NR³.

19. The compound according to claim 18, wherein Z² is optionally substituted C₂₋₄alkylene or optionally substituted phenylene wherein the optional substituents are selected from one or two of halo, C₁₋₄alkyl, fluoro-substituted-C₁₋₄alkyl, phenyl and fluoro-substituted phenyl.

20-21. (canceled)

22. The compound according to claim 18, wherein Z² is propylene.

23. The compound according to claim 1, wherein the compound of Formula I is selected from:

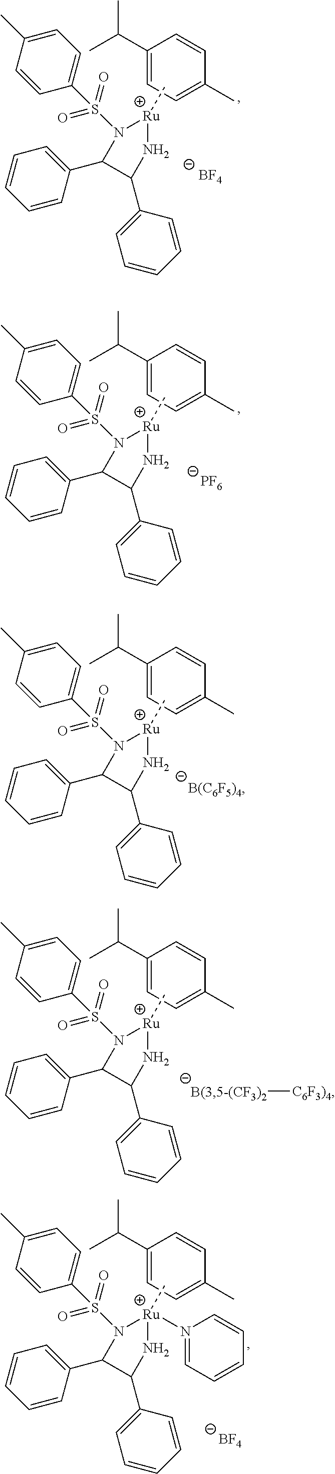

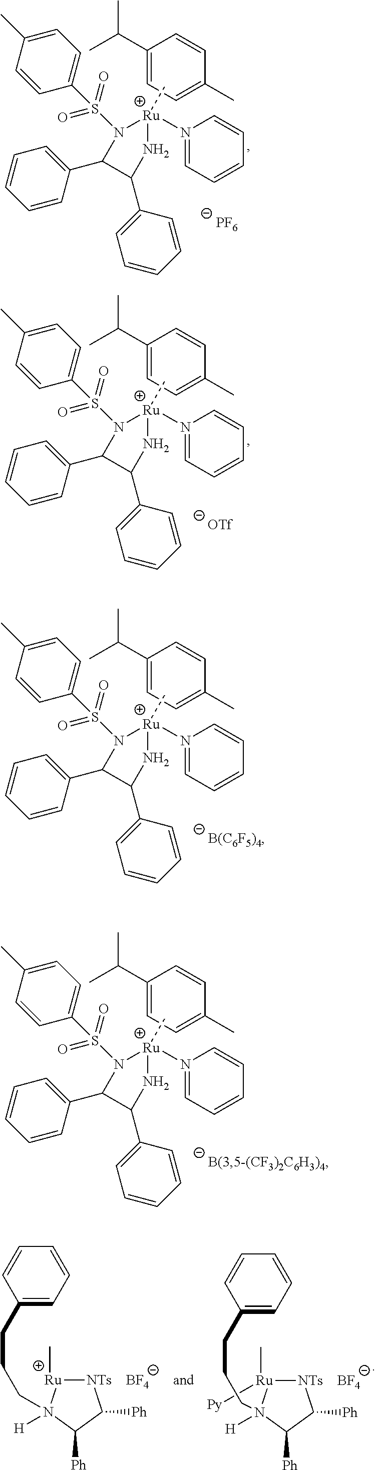

24. A process for preparing a compound of Formula I according to any one of claims 1-23 comprising combining a precursor ruthenium compound, an anion abstracting agent, a compound of the Formula D-Z¹—NHR¹ wherein D, Z¹ and R¹ are as defined in claim 1, and optionally a base and reacting under conditions to form the compound of Formula I and optionally, isolating the compound of Formula I.

25-26. (canceled)

27. A process for the reduction of compounds comprising one or more carbon-oxygen (C═O) or carbon-nitrogen (C═N) double bonds, to the corresponding hydrogenated alcohol or amine, comprising contacting a compound comprising the C═O or C═N double bond with a compound of the Formula I according to claim 1 under transfer hydrogenation conditions.

28-30. (canceled)
